(19) United States
(12) Patent Application Publication
(10) Pub. No.: US 2016/0067035 A1
(43) Pub. Date: Mar. 10, 2016

(54) Intraocular Lens With Central Hole For Improved Fluid Flow And Minimized Light Scattering

(71) Applicants: Ivair Gontijo, Los Angeles, CA (US); Thomas R. Paul, Westlake, CA (US); Alexei Ossipov, Laguna Niguel, CA (US)

(72) Inventors: Ivair Gontijo, Los Angeles, CA (US); Thomas R. Paul, Westlake, CA (US); Alexei Ossipov, Laguna Niguel, CA (US)

(73) Assignee: STAAR Surgical Company, Monrovia, CA (US)

(21) Appl. No.: 14/849,382

(22) Filed: Sep. 9, 2015

Related U.S. Application Data
(60) Provisional application No. 62/048,007, filed on Sep. 9, 2014.

(51) Int. Cl. A61F 2/16; B23B 51/00

(52) U.S. Cl. CPC A61F 2/1601; B23B 51/0081; A61F 2002/1681; A61F 2002/1696

(57) ABSTRACT

An implantable contact lens having a central hole with angled walls optimized to minimize light scattering is described. The central hole provides fluid flow from the posterior to the anterior chamber of the eye, and its shape and size of designed to reduce glare and halos resulting from light scattered by the walls of the hole. The design parameters of the hole are dependent on the refractive index of the material in which the central hole is formed.

### CROSS-REFERENCES TO RELATED APPLICATIONS

This application claims priority from U.S. Provisional Application No. 62/048,007, filed Sep. 9, 2014, incorporated by reference in its entirety.

### BACKGROUND

The invention is generally directed to improvements in the functioning of an intraocular lens or other type of ocular implant where fluid flow between a posterior side of the implant and an anterior side of the implant is necessary. More specifically, the invention provides an improved central fluid passageway that also minimizes the scattering of light by the central fluid passageway.

There are several optical conditions for which correction is typically desired, if not required. Examples of such conditions include myopia, hyperopia and presbyopia. Today, several solutions for these conditions are known. The simplest one is the use of glasses to provide corrected vision. Although this solution works well, there are situations when glasses are inconvenient or not recommended. For aesthetic reasons many persons would prefer to use a less obvious vision correction method.

Traditionally, contact lenses have also been used to correct a person's vision where the person has desired to forego the use of glasses. Contact lenses, however, may be difficult to insert and remove, and may also not be able to fully correct a person's visual problems.

Refractive surgical solutions have been developed to correct visual abnormalities and to improve a person's vision without requiring the use of glasses or contact lenses. For example, one surgical solution is LASIK (laser assisted in situ keratomileusis), which involves the ablation of internal parts of the cornea to provide optical correction. LASIK is a good solution for correction, but may not be appropriate for everyone. For example, LASIK is not recommended for people who have very thin corneas, on the order of 0.5 mm center thickness or less. In addition, if the eye changes with ageing, it is not possible to repeat the surgery several times because it is a subtractive solution, where material is removed from the cornea.

An additional disadvantage of a subtractive surgical procedure is that it is not completely reversible, that is, once the subtractive procedure has been done on the eye it is not possible to bring the eye back to its original state prior to the surgery if the person requires such a reversal, or even if the person for some reason desires to have their old vision back.

Implantable contact lenses on the other hand have advantages over previous solutions, as they can be implanted and explanted if needed. However, implantable contact lenses may cause unequal pressure to form between the anterior and posterior chambers of the eye. One solution includes an intraocular lens with a central hole to equalize the pressure between the anterior and posterior chambers of the eye. For further details, see U.S. Pat. No. 5,913,898. Although this solution works well to equalize the pressure between the anterior and posterior chamber of the eye, the described hole is not optimized to reduce light scattering by the internal walls of the hole. As a result, in some instances, light falling upon the hole in this lens would scatter light onto the retina of the eye, occasionally causing the person in whom the lens had been implanted to report seeing arcs and halos.

What has been needed, and heretofore unavailable, is an intraocular contact lens having a central hole for providing fluid flow between the anterior and posterior chambers of an eye in which the intraocular contact lens is implanted. The shape and size of the improved hole are configured to minimize the light scattered from the walls of the hole, thus reducing the incidence of halos, arcs or other visual aberrations caused by the scattered light. The present invention satisfies these, and other needs.

### SUMMARY

In its most general aspect, the present invention includes an implantable intraocular contact lens having a central hole with angled or tilted walls. The central hole allows fluid flow from the posterior to the anterior chamber of the eye through the intraocular contact lens and at the same time solves a serious problem present in previous lenses with holes that had vertical walls. Light scattered by the vertical walls in previous lenses form luminous arcs on the retina that are perceived by the lens wearer as glare and halos. The tilted walls of the central hole of this invention prevent the hole from forming these arcs and focuses the light scattered by the hole on the same spot as the rest of the lens optic. The hole may have a range of diameters, such as for example, from 0.05 millimeters to 0.40 millimeters, and tilt angles from 5 degrees to 75 degrees, depending on the specific refractive index of the material being used to form the intraocular contact lens.

In another aspect, the present invention includes an intraocular contact lens for implantation into an eye, comprising: a body portion surrounding an optical zone, the optical portion having a thickness and an optical axis transverse to a longitudinal axis of the body portion; and a hole disposed in the optical zone extending through the thickness of the optical zone from an anterior side of the optical portion to a posterior side of the optical zone, the hole having a wall formed by the thickness of the optical zone, the hole wall being angled relative to the optical axis such that the anterior surface diameter of the hole is different than the posterior surface diameter of the hole. In one alternative aspect the anterior surface diameter of the hole is smaller than the posterior surface diameter of the hole. In another alternative aspect, the anterior surface diameter of the hole is larger than the posterior surface diameter of the hole.

In yet another aspect, the hole wall is angled relative to the optical axis in range of 5 degrees to 75 degrees. In another aspect, the hole wall is angled 65 degrees relative to the optical axis. In another alternative aspect, the hole wall is angled 65 degrees relative to the optical axis and the anterior surface diameter of the hole is smaller than the posterior surface diameter of the hole.

In still another aspect, the wall of the hole has a curvature extending between the anterior surface of the optical zone and the posterior surface of the optical zone. In one aspect, the curvature has a 2.0 millimeter radius.

In an alternative aspect, the wall of the hole further comprises an annular portion extending from the anterior surface of the optical zone for a selected distance to an endpoint and a tapered portion extending from the endpoint to the posterior surface of the lens. In one alternative aspect, the diameter of the hole within the annular portion is smaller than the diameter of the hole at the posterior surface of the lens.

In a further aspect, the hole wall has a step-like profile, with each step having a larger diameter than the next adjacent step moving in the direction from the step having the smallest diameter to the step having the largest diameter.

In a still further aspect, the hole is disposed in a center of the optical portion. In another aspect, there may be a plurality of holes formed in the optical portion of the lens, or they may be formed at or adjacent to a transition between the optical portion and the body portion of the lens.

In still another aspect, there may be a plurality of holes formed in the body portion of the lens, or they may be formed at or adjacent to a transition between the optical portion and the body portion of the lens.

In yet another aspect, the hole has a configuration that is optimized to reduce the amount of light scattered by the wall of the hole onto a retina.

In still another aspect, the present invention includes a method of forming a hole configured to reduce the amount of light scattered by the wall of the hole in the optical zone of an intraocular contact lens, comprising: drilling a tapered hole through an optic zone of the intraocular contact lens having an anterior surface and a posterior surface such that the hole as a first diameter at the anterior surface of the optic zone and a second diameter at the posterior surface of the optic zone. In another aspect, the tapered hole is configured to reduce light scattered by a wall of the tapered hole.

Other features and advantages of the invention will become apparent from following detailed description taken in conjunction with the accompanying drawings, which illustrate, by way of example, the features of the invention.

### BRIEF DESCRIPTION OF THE DRAWINGS

FIG. 1 is a top view on one embodiment of a prior art intraocular contact lens having a hole located in the center of its optic zone to provide fluid flow between an anterior and posterior sides of the intraocular lens

FIG. 2 is a cross-sectional side view of the embodiment of FIG. 2 illustrating the details of the hole located in the center of the optic zone.

FIG. 3 is a cross-sectional side view of one embodiment of an intraocular contact lens similar to that of FIG. 1 except that the sides of the central hole are angled from the anterior side of the lens to the posterior side of the lens in accordance with principles of the present invention.

FIG. 4A is a graphical representation of a ray trace analysis performed on a lens having a serrated hole.

FIG. 4B is a perspective view of a lens having a serrated hole, the hole having a step wise appearance.

FIG. 5A is a graphical representation of a ray trace analysis performed on a hole with walls tilted by 45 degrees from the anterior surface of the lens to the posterior surface of the lens.

FIG. 5B is a graphical representation of a ray trace analysis performed on a hole with walls tilted by 45 degrees from the posterior surface of the lens to the anterior surface of the lens.

FIG. 6 is a cross-sectional view of an intraocular lens in accordance with the present invention having a draft angle of 45 degrees relative to the optical axis of the lens.

FIG. 7A is a cross-sectional view of an ICL having a central hole having an annular portion disposed adjacent the anterior surface of the lens.

FIG. 7B is an enlarged cross-sectional view the ICL of FIG. 7A showing the detail of the hole.

FIG. 8A is a graphical representation of a ray trace analysis showing a case where all light reaches the retina.

FIG. 8B is a graphical representation of a ray trace analysis showing only rays that hit the annular portion of the hole wall of FIGS. 7A-B.

FIG. 9A is a cross-sectional view of an ICL having a hole having a radiused wall portion.

FIG. 9B is an enlarged cross-sectional view of the ICL of FIG. 9A showing details of the radiused wall.

FIG. 9C is a graphical representation of a ray trace analysis showing light scattering of by the hole FIGS. 9A-B.

FIG. 10A illustrates a first face of an ICL used during a ray trace analysis of the ICL.

FIG. 10B illustrates a second face of the ICL used during a ray trace analysis of the ICL.

FIG. 10C illustrates a third face of the ICL used during a ray trace analysis of the ICL.

FIG. 11A is a graphical representation of a ray trace analysis showing a side view of the rays hitting the retina.

FIG. 11B is a graphical representation of a ray trace analysis showing a front view of rays hitting the retina and forming arcs on the retina due to rays scattered by the wall of the hole in the center of the lens of FIGS. 10A-C.

FIG. 12A is a graphical representation of a ray trace analysis showing a side view of the rays hitting the retina scattered by the hole.

FIG. 12B is a graphical representation of a ray trace analysis showing a front view of rays hitting the retina and forming arcs on the retina due to rays scattered by the wall of the hole in the center of the lens of FIGS. 10A-C. Some of the rays are reflected off the internal wall of the hole without hitting the optical zone, other rays pass through the optical zone first and then hit the hole wall from the lens side and then undergo total internal reflection.

FIG. 13A is a graphical representation of a ray trace analysis showing a side view of the rays hitting the retina.

FIG. 13B is a graphical representation of a ray trace analysis showing a front view of rays hitting the retina and forming arcs on the retina due to rays scattered by the wall of the hole in the center of the lens of FIGS. 10A-C that first hit the optic zone and then hit the hole wall, where they undergo total internal reflection.

FIG. 14A is a graphical representation of a ray trace analysis showing a side view of the rays hitting the retina.

FIG. 14B is a graphical representation of a ray trace analysis showing a front view of rays hitting the retina and forming arcs on the retina due to rays scattered by the wall of the hole in the center of the lens of FIGS. 10A-C that first hit the internal wall of the hole, but do not hit the optic.

FIG. 15A is a graphical representation of a ray trace analysis showing a side view of the rays hitting the retina.

FIG. 15B is a graphical representation of a ray trace analysis showing a front view of rays hitting the retina and forming arcs on the retina due to rays scattered by the wall of the hole in the center of the lens of FIGS. 10A-C, showing only the rays that hit the retina passing through the optical zone or the central hole without hitting anything.

FIG. 16A is a graphical representation of a ray trace analysis showing a side view of the rays hitting the retina.

FIG. 16B is a graphical representation of a ray trace analysis showing a front view of rays hitting the retina and forming arcs on the retina due to rays scattered by the wall of the hole in the center of the lens of FIGS. 10A-C showing only the rays passing through the central hole without hitting anything.

FIG. 17 is a graphical representation of a ray trace analysis showing a front view of rays hitting the retina and forming arcs on the retina due to rays scattered by the wall of the hole in the center of the lens of FIGS. 10A-C.

FIG. 18A is a graphical representation of a ray trace analysis showing a side view of the rays hitting the retina.

FIG. 18B is a graphical representation of a ray trace analysis showing a front view of rays hitting the retina and forming arcs on the retina due to rays scattered by the wall of the hole in the center of the lens of FIGS. 10A-C.

FIG. 19 is a plot of peak irradiance as a function of hole diameter.

FIG. 20A is a graphical representation of a ray trace analysis showing a front view of rays hitting the retina for a no hole model.

FIG. 20B is a graphical representation of a ray trace analysis showing a front view of rays hitting the retina and forming arcs on the retina due to rays scattered by the wall of the hole in the center of the lens for a hole having straight walls.

FIG. 21A is a graphical representation of a ray trace analysis showing a front view of rays hitting the retina and forming arcs on the retina due to rays scattered by the wall of the hole in the center of the lens for a hole tilted 5 degrees.

FIG. 21B is a graphical representation of a ray trace analysis showing a front view of rays hitting the retina and forming arcs on the retina due to rays scattered by the wall of the hole in the center of the lens for a hole tilted 10 degrees.

FIG. 22A is a graphical representation of a ray trace analysis showing a front view of rays hitting the retina and forming arcs on the retina due to rays scattered by the wall of the hole in the center of the lens for a hole tilted 15 degrees.

FIG. 22B is a graphical representation of a ray trace analysis showing a front view of rays hitting the retina and forming arcs on the retina due to rays scattered by the wall of the hole in the center of the lens for a hole tilted 35 degrees.

FIG. 23A is a graphical representation of a ray trace analysis showing a front view of rays hitting the retina and forming arcs on the retina due to rays scattered by the wall of the hole in the center of the lens for a hole tilted 45 degrees.

FIG. 23B is a graphical representation of a ray trace analysis showing a front view of rays hitting the retina and forming arcs on the retina due to rays scattered by the wall of the hole in the center of the lens for a hole tilted 55 degrees.

FIG. 24A is a graphical representation of a ray trace analysis showing a front view of rays hitting the retina and forming arcs on the retina due to rays scattered by the wall of the hole in the center of the lens for a hole tilted 65 degrees.

FIG. 24B is a graphical representation of a ray trace analysis showing a front view of rays hitting the retina and forming arcs on the retina due to rays scattered by the wall of the hole in the center of the lens for a hole tilted 75 degrees.

FIG. 25A is a graphical representation of a ray trace analysis showing the layout of the model in a “no hole” case.

FIG. 25B is a graphical representation of a ray trace analysis showing the MTF plot for the lens in FIG. 17.

FIG. 26A is a graphical representation of a ray trace analysis showing the layout of the model in a hole tilted 0 degrees.

FIG. 26B is a graphical representation of a ray trace analysis showing the MTF plot for the lens in FIG. 26A.

FIG. 27A is a graphical representation of a ray trace analysis showing the layout of the model in a hole tilted 55 degrees.

FIG. 27B is a graphical representation of a ray trace analysis showing the MTF plot for the lens in FIG. 27A.

FIG. 28A is a graphical representation of a ray trace analysis showing the layout of the model in a hole tilted 65 degrees.

FIG. 28B is a graphical representation of a ray trace analysis showing the MTF plot for the lens in FIG. 28A.

FIG. 29A is a graphical representation of a ray trace analysis showing the layout of the model in a hole tilted 75 degrees.

FIG. 29B is a graphical representation of a ray trace analysis showing the MTF plot for the lens in FIG. 29A.

FIG. 30A is a graphical representation of a ray trace analysis showing light scattered using light having an incidence of 5 degrees for a lens having a hole wall tilted at 75 degrees.

FIG. 30B is a graphical representation of a ray trace analysis showing light scattered using light having an incidence of 15 degrees for a lens having a hole wall tilted at 75 degrees.

FIG. 31A is a graphical representation of a ray trace analysis showing light scattered using light having an incidence of 25 degrees for a lens having a hole wall tilted at 75 degrees.

FIG. 31B is a graphical representation of a ray trace analysis showing light scattered using light having an incidence of 35 degrees for a lens having a hole wall tilted at 75 degrees.

FIG. 32 is a graphical representation of a ray trace analysis showing light scattered using light having an incidence of 45 degrees for a lens having a hole wall tilted at 75 degrees.

### DETAILED DESCRIPTION OF THE EMBODIMENTS

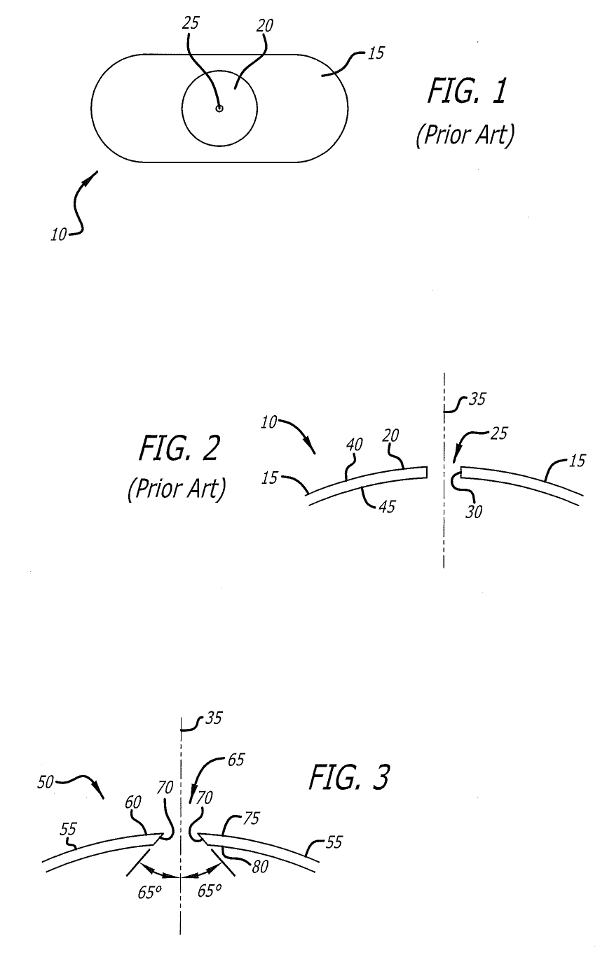

Referring now to the drawings in detail, in which like reference numerals indicate like or corresponding elements among the several figures, there is shown in FIG. 1 and embodiment of a prior art intraocular contact lens 10 having an oval shaped body having a peripheral portion 15 and an optical zone or portion 20. This type of intraocular contact lens is designed to be placed within a phakic eye between a person's crystalline lens and the iris. One such lens is described in U.S. Pat. No. 5,913,898, which is intended to be incorporated herein in its entirety.

A hole 20 is disposed in the center of the optical zone to provide for fluid flow between an anterior side of the lens (shown in the top view) and a posterior side of the lens (not shown). Providing fluid flow between the anterior and posterior sides of the lens in this manner provides for equalization of pressure between the sides of the lens, thus preventing possible interference with the operation of the iris of the eye, which may damage the iris and result in increased intraocular pressure of the eye.

FIG. 2 is a cross-sectional view of the lens of FIG. 1. In this view, axis 35 has been added so that details of hole 25 may be described. As can be seen, hole 25 in the prior art lens was formed in the optic zone of the lens in such a manner that walls 30 of the hole are parallel to axis 35. While this arrangement works well for its intended purpose of providing fluid flow between anterior side 40 and posterior side 45 of the lens, in some cases, aberrations caused by light refracted by the sides of the hole may be visible to the person in which the lens is implanted.

FIG. 3 is a cross-sectional view of an exemplary embodiment of the present invention illustrating an improvement to the prior art wherein the hole in the central optic of the intraocular contact lens is formed so that the wall of the hole are no longer parallel to axis 35, but instead are angled, or tilted with respect to axis 35. In this embodiment, walls 70 are angled in relation to axis 35 by an angle Φ of 65 degrees from anterior surface 75 of the optical zone 60 to posterior surface 80 of the optical zone.

As will be discussed in more detail below, the optimal tilt angle of the walls of the hole is dependent on the size of the hole and the refractive index of the lens material. While hole size has been found to affect the optical scattering caused by the walls of the hole, in one embodiment of a lens manufactured from a Collamer material (Collamer is a registered trademark of STAAR Surgical Company) having an index of refraction of 1.441, reduction in aberrations such as arcs and halos was optimized for a hole diameter of 300 microns with a wall tilt from anterior to posterior surface of the lens of 65 degrees.

While walls 70 of hole 65 are angled at 65 degrees in this embodiment, other alternative arrangements and angles are possible, as will be described in more detail below. For example, the hole may have a diameter of between 50 microns and 400 microns, and still provide for adequate fluid flow between the anterior and posterior surfaces of the lens, and, as will be shown below, the optical performance of the lens may still be optimized by adjusting the angle of the tilt of the walls of the hole. For example, in one embodiment in which the Collamer material described above is used, the walls may be tilted within a range of 50 degrees and 75 degrees to provide for reduced arcs and halos. As noted above, however, the optimal hole sizes and ranges will depend on the index of refraction of the material used to manufacture the intraocular contact lens. As one skilled in the art will immediately understand, changing the index of refraction of the material used will result in different optical performance of the lens, including how light incident on the walls of the hole is refracted by the walls of the hole.

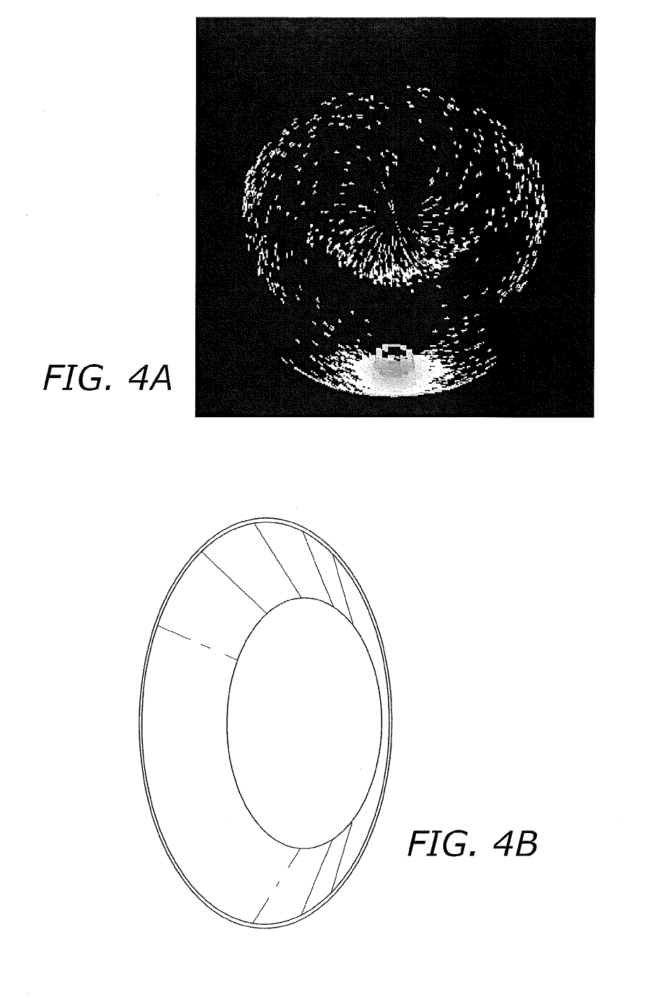

Other configurations of the tilted wall will similarly function to reduce the amount of optical aberrations caused by light refracting from the walls of the hole in the center of the optical zone of the lens. For example, as shown in FIG. 4, the hole in the center of the optic zone may be created in a manner that results in a step-wise increase in the diameter of the hole from anterior surface of the lens to the posterior surface of the lens. Such a step-wise increase in diameter results in hole having a “serrated” appearance when viewed from the posterior side of the lens. A hole formed in this manner reduces the peak irradiance of the retina by spreading the light over the retina. In this case, when the peak irradiance is below a certain threshold the aberrant light will not be visible to the person in which the lens is implanted.

In the example illustrated in FIG. 4, the overall angle of the walls of the hole (assuming smoothing of the serrations) is 45 degrees. As can be seen in the graph generated in accordance with the procedures discussed in more detail below, the peak irradiance for the light coming from the hole is only 0.396 W/cm², compared to 131.57 W/cm² for the light passing through the optic. Thus, the scattering coming from the hole represents only 0.3% of the total irradiance reaching the cornea in the model eye.

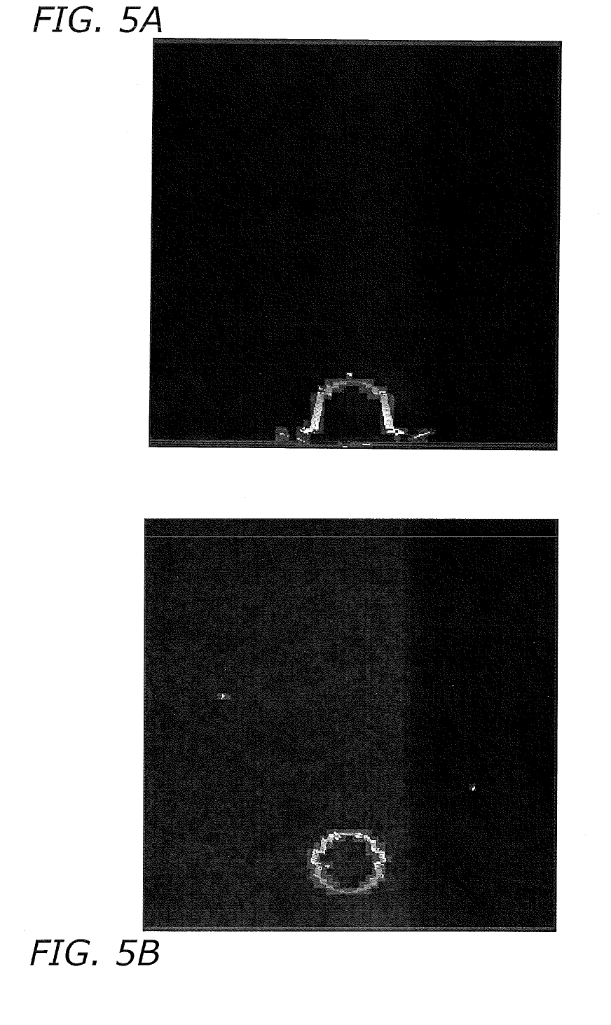

FIGS. 5A-B illustrate that the angle of the walls of the hole may also be tilted from the posterior surface of the lens to the anterior surface of the lens and still provide for reduced aberrations. In such a lens design, the diameter of the hole increases from the posterior surface of the lens to the anterior surface of the lens. This is shown by comparing the scattering of light from a hole that includes walls that are angled from anterior to posterior (FIG. 5A) to the light scattered by the walls of a hole, wherein the walls are angled from posterior to anterior (FIG. 5B).

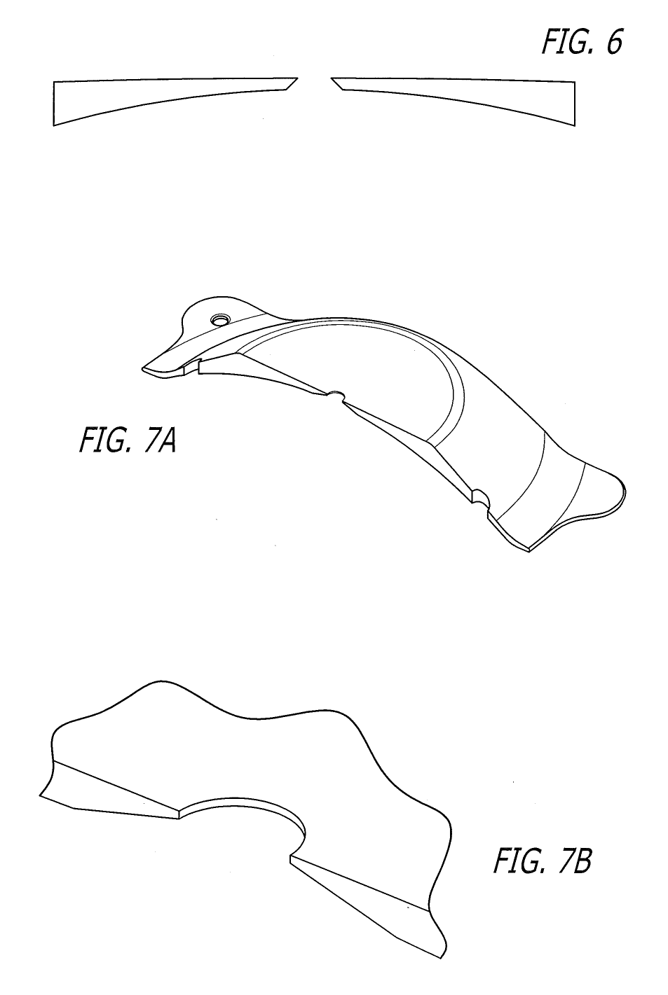

FIG. 6 is a cross-sectional view illustrating an alternative embodiment of the present invention having an anterior surface radius of 100 millimeters and a posterior surface radius of 10.401 millimeters. The diameter of the central hole of this lens is 0.360 millimeters and hole walls are tilted by 45 degrees from the anterior surface to the posterior surface of the lens. As can be seen in this view, the tilt of the walls of the hole begins at the anterior surface of the lens, resulting in a hole having no thickness in the vertical direction that parallels the optical axis of the lens.

Such a hole may be manufactured using various types of tools. For example, a drill with a diameter slightly smaller than the hole final diameter may be used first, followed by a second tool with a conical shape that produces the tilted walls and the final hole diameters. In such a case, variations in the final hole diameter may exist, or some material of the vertical wall might be left un-removed. One improvement to this process may be to produce a hole as shown in FIG. 7A.

FIG. 7A is a cross-sectional perspective view of an intraocular contact lens where the hole is formed in a manner which leaves some material in the vertical wall before the angle of the wall begins. This configuration is illustrated in FIG. 7B, which shows an annulus of material having a thickness that is measured from the anterior surface of the lens and extends 0.020 millimeters, at which point the angled portion of the hole begins.

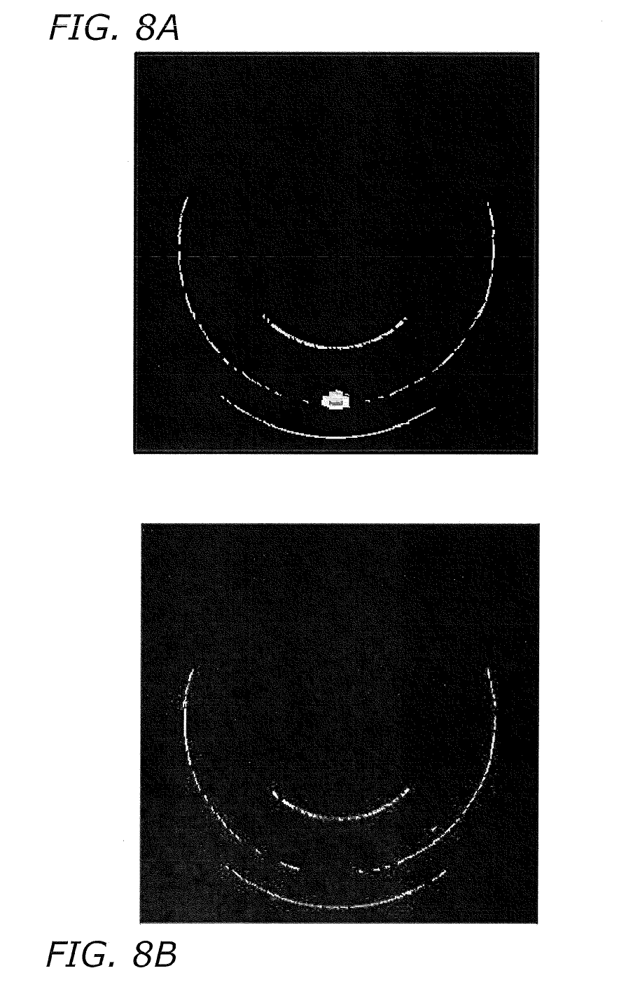

FIGS. 8A and B compare the scatter of light in the case where all of the light reaches the retina (FIG. 8A) and the case where some of light is scattered by the 0.020 millimeter wide annulus of the hole of FIG. 7A. In FIG. 8A, the total radiance reaching the retina is 131.46 W/cm² and the irradiance due to rays scattered by the .020 millimeter wall is 1.6108e⁻² W/cm². Thus, only about 0.01% of the total irradiance reaching the retina would be due to the light scattered by the 0.020 millimeter annulus. Such a small level of scattered light may not be visible to a person. FIG. 8B illustrates the embodiment in which some of light is scattered by the 0.020 millimeter wide annulus of the hole of FIG. 7A

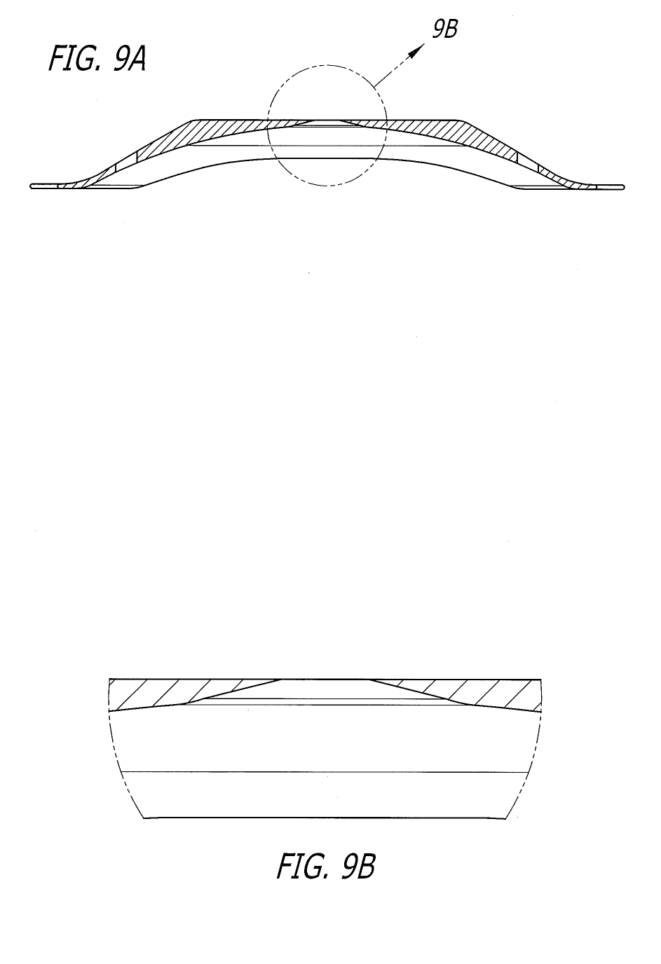

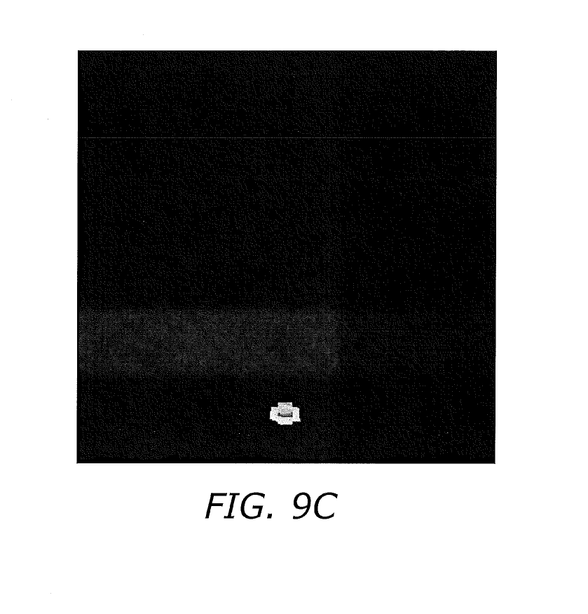

FIG. 9A is a cross-sectional view of another alternative embodiment of the present invention. As shown in FIG. 9B, an enlarged view of the central hole of FIG. 9A, the hole is formed in a manner such that instead of the wall being linear, the wall has small radius, such as, for example, 2.0 millimeter. Forming the wall in this manner changes the optical power of the this area of the lens, resulting in a bi-focal lens.

FIG. 9C is a graph illustrating the light scattered by the curved walls. As can be seen, the light is focused in the same area of the model retina as for straight wall embodiments, indicating that the curved walls do not produce arcs and halos. In this model, light is incident at 35 degrees.

It will be apparent to those skilled in the art that the various features described above can be combined and are intended to be within the scope of the present invention. For example, other holes may be added to the lens outside of the optic area to avoid any possibility of occlusion and increase in eye pressure. Similarly, instead of a single hole at the center of the lens, one or more small holes may be placed near the periphery of the optic zone of the lens, providing essentially the same function of improving fluid flow, while reducing the amount of scattered light.

### Description of Testing and Model Eye

Extensive simulations of light scattering by the central hole in intraocular contact lenses to optimize the reduction in light scattering caused by the central holes of prior art lenses were performed. Zemax 13 Release 2 SP1 Professional (64 bits) software operating on a computer system having sufficient memory and processor power was used in these simulations. The results of the simulations were displayed using various colors to assist in the differentiation of various optical effects that were produced by the simulation. Obviously, these color representations cannot be reproduced in black and white print, and every effort has been made to describe the effects in a manner which will be familiar to a skilled person.

In the simulations, the central hole could be varied in diameter from 100 microns (0.1 millimeters) to 400 microns (0.4 millimeters) and the walls of the hole could be beveled. The draft angle of the hole walls was varied from zero degrees (straight hole) up to 75 degrees and the effect of these hole shape changes on the amount of scattered light was studied.

A light beam having an intensity of 1 watt was divided into 5,000,000 rays (that is, each ray carried 200 nW of power). In the simulation, most of the light is stopped by the sclera and iris of the model eye. Those rays that pass through the iris of the model eye used in the simulation then pass through the intraocular contact lens (ICL) or hit the internal walls of the central hole of the ICL. In both cases, these rays proceed through the crystalline lens of the model eye and fall upon the retina of the model eye. As described above, holes of smaller diameters are possible also, as the minimum hole size to produce adequate flow and equalize the pressure between the anterior and posterior sides of the ICL has been found to be only 50 microns (0.05 millimeters). Thus the 100 um (0.1 millimeter) hole is sufficient large as to provide a margin against occlusion and pressure increases. Additionally, a hole larger than 400 um (0.4 millimeters) diameter is also possible, but such a large diameter hole may adversely affect the lens Modulation Transfer Function (MTF) of the ICL. A draft angle larger than 75 degrees could be used as well, if the center part of the lens affected by the hole is given a different radius of curvature, as discussed herein.

The starting case for the simulation was an ICL manufactured from a Collamer material having a central hole, implanted in the Liou & Brennan (LB) model eye described in “Anatomically accurate, finite model eye of optical modeling”, Hwey-Lan Liou and Noel A. Brennan, J. Opt. Soc. Am. A, Vol. 14, No. 8, August 1997, 1684, which hereby incorporated herein in its entirety. As the original LB model is emmetropic, it was modified by increasing the vitreous humor thickness (the eye depth) so that light is properly focused on the retina of the simulated eye when the lens is implanted.

Another change made to the LB eye model was the replacement of the gradient index crystalline lens with a lens having a constant refractive index and the same optical power. This simplifies the calculations of light scattering in NSC mode in Zemax and has no effect on the final result, as neither the original LB lens nor the modified LB lens produce any scattered light. Either lens is used simply to focus the light on the retina.

In the preferred model used during the simulations described herein (unless otherwise indicated), the incident light comes into the model eye at an angle of 35 degrees, which represents a worst case scenario for light scattering as described by Holladay et. al. in “Analysis of edge glare phenomena in intraocular lens edge designs,” Jack T. Holladay, MD, MSEE, Alan Lang, PhD, Val Portney, PhD, J. Cataract Refract. Surg. 25, 748-752, 1999, which is hereby incorporated herein in its entirety.

Alternatively, the hole shape and size for light coming in at zero degrees or some other incidence angle may be simulated to optimize the performance of the ICL. A Computer Aided CAD model of the ICL lens design to be tested was imported into the Zemax optical ray tracing software and different faces of the lens were defined, allowing rays that hit each face to be analyzed separately.

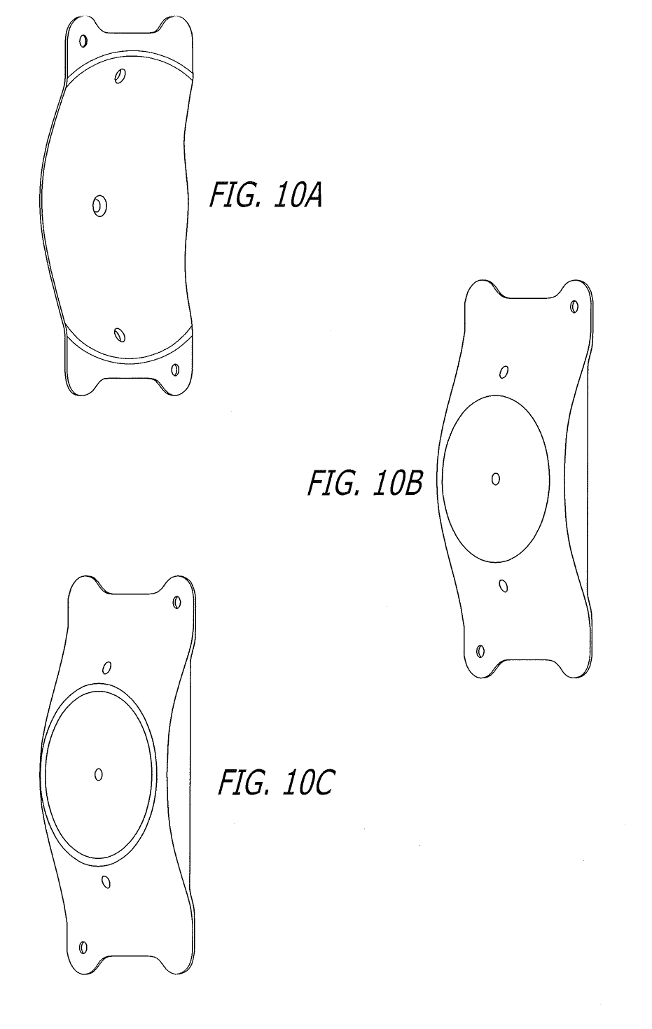

As shown in FIGS. 10A-C, The lens was divided into 4 faces. FIG. 10A depicts the posterior side or surface of the lens, and highlights the internal surface of the central hole. FIGS. 10B-C depict the anterior side of the lens. FIG. 10B highlights the anterior surface of the optic zone, and FIG. C highlights the transition ring that surrounds the optic zone and is located between the optic zone and the haptics of the lens.

The results of the various simulations carried out during the testing will be summarized in the following terms:

The light scattering results are summarized in terms of:

Number of hits: the number of rays hitting the retina, coming from a particular face. Each ray (each hit) carries 200 nW of power. A skilled person would understand that it would be trivial to change this setting to make each ray carry a different amount of power;

Power: the optical power corresponding to the rays hitting the retina (or 200 nW×Number of Hits);

Peak Irradiance: Irradiance or power density is more important than power. For example, if 100 microwatts of power is concentrated in a small area of the retina, it will be perceived. On the other hand, if the light is spread out over a large area of the retina, the resulting power of the light reaching each light sensor on the retina might be too small and not register as perceived. Peak Irradiance (or power per unit area) is the quantity that measures power density.

In the first analysis, the light scattering was analyzed to determine which rays came from which face of the lens. In these tests, lenses with holes of different shapes and sizes were analyzed. All lenses used in the tests had optical power of −10.0D, unless otherwise specified. Those skilled in the art will recognize that other optical powers will behave similarly. In these tests, the lenses were made of Collamer, but as previously described, could be made of any other suitable material, correcting for the refractive index of the material.

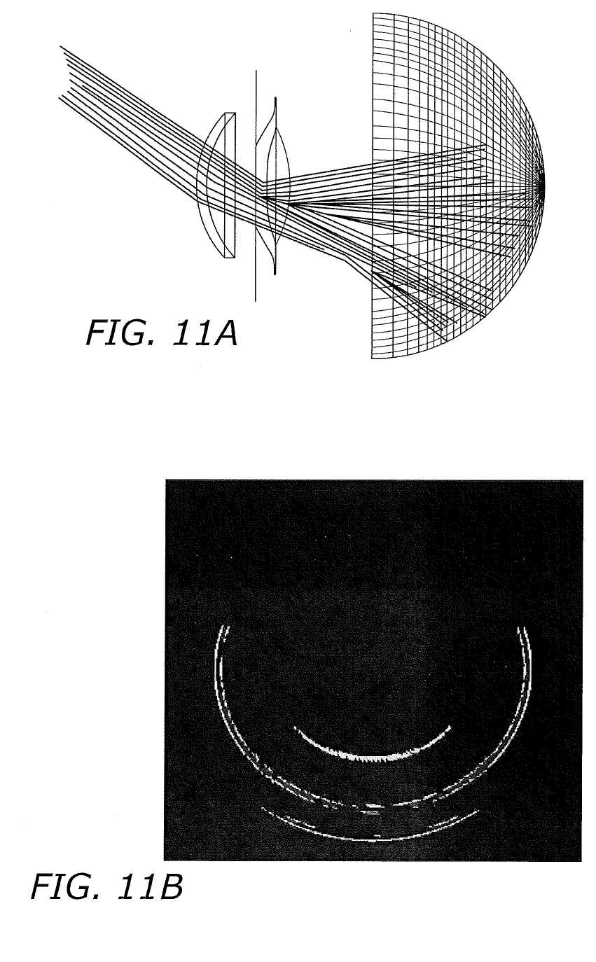

FIGS. 11A-B provide a side and front view of the LB model eye with an ICL implanted in front of the crystalline lens. The light entering the model eye is incident at 35 degrees. Referring to FIG. 11A, a ray trace analysis showing a side view of the rays hitting the retina when the entering light is incident at 35 degrees is shown. In particular, the illustration shows all of the rays that hit the retina, as seen from the side of the model eye. Referring to FIG. 11B, a ray trace analysis illustrating the rays of FIG. 11A hitting the retina from a front view. In particular, the illustration shows the rays looking down at the retina. In this view, there is a bright spot that is produced by the focus of all the rays coming from optical zone of the lens. The arcs that are seen are the result of rays that are scattered by the wall of the central hole.

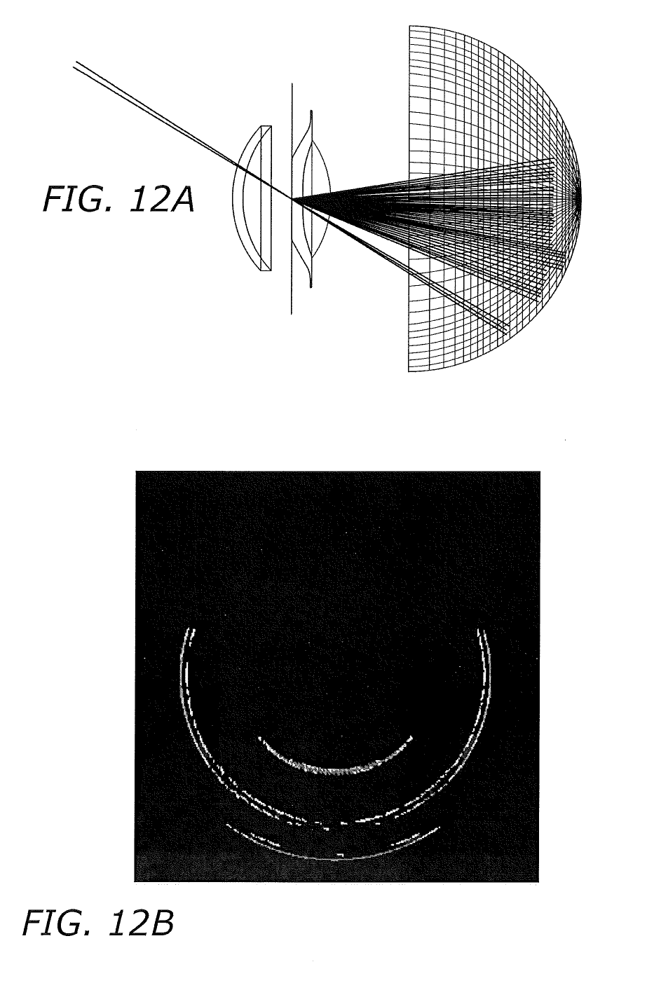

FIGS. 12A-B illustrate the results of the testing where the simulation is filtered so that only rays that hit the hole are shown. Referring to FIG. 12A, a ray trace analysis showing a side view of the rays hitting the retina is shown. In particular, the illustration shows the rays scattering from hole from the side. Referring to FIG. 12B, a ray trace analysis illustrating the rays of FIG. 12A hitting the retina from a front view is shown. There are two types of rays that hit the wall of the hole. Some reflect off the internal wall of the hole without then passing through the optical zone. Other rays pass through the optical zone first and then hit the wall of the hole from the lens side and then undergo total internal reflection. As shown in FIG. 12B, one group of rays forms the lowest arc while the other forms the two higher arcs.

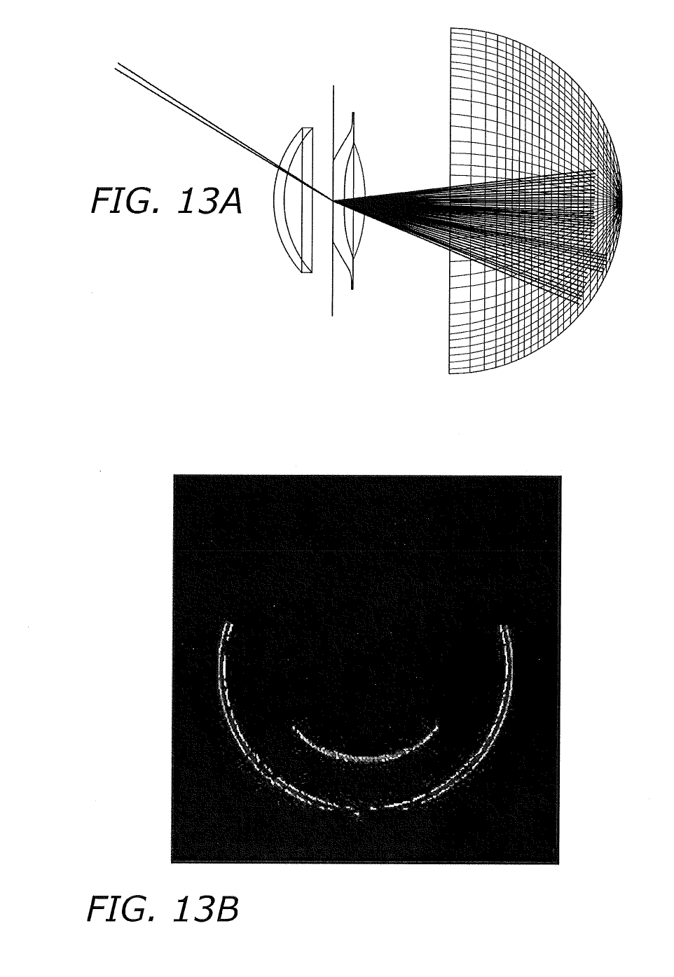

FIGS. 13A-B further filter the results of the testing described above, showing only the rays that first hit the optical zone and then hit the wall of the hole, where they undergo total internal reflection, forming the top group of arcs and halos. Referring to FIG. 13A, a ray trace analysis showing a side view of the rays hitting the retina is shown. Referring to FIG. 13B, a ray trace analysis illustrating the rays of FIG. 13A hitting the retina from a front view is shown.

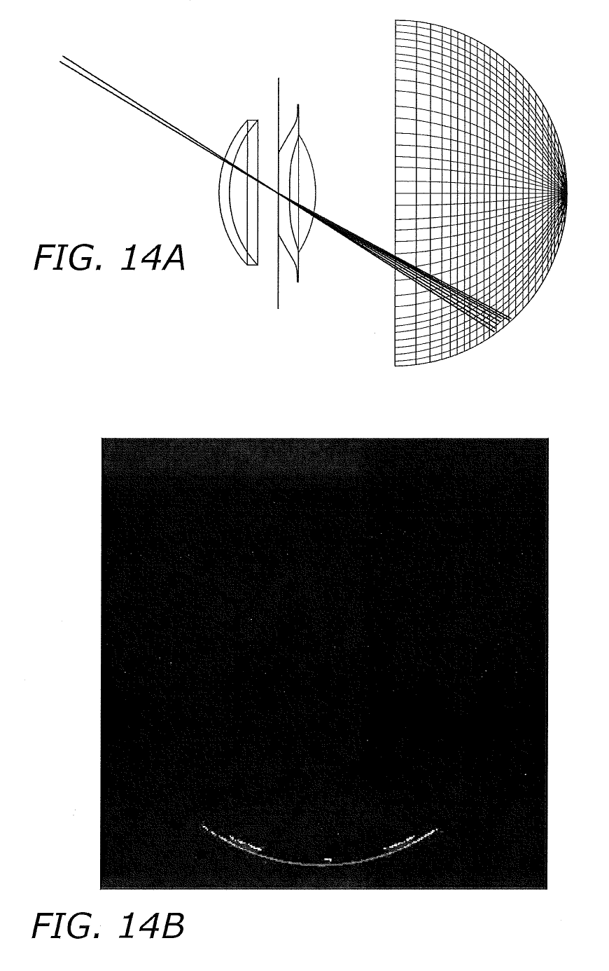

FIGS. 14A-B further filter the results, selecting only the rays that hit the internal wall of the hole, but do not hit the optical zone. An arc can be seen in FIG. 14B that is formed by these rays. Referring to FIG. 14A, a ray trace analysis showing a side view of the rays hitting the retina is shown. Referring to FIG. 14B, a ray trace analysis illustrating the rays of FIG. 14A hitting the retina from a front view is shown.

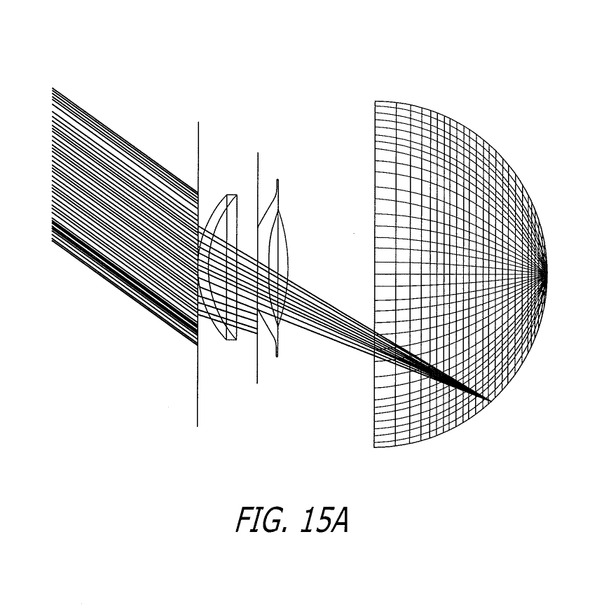

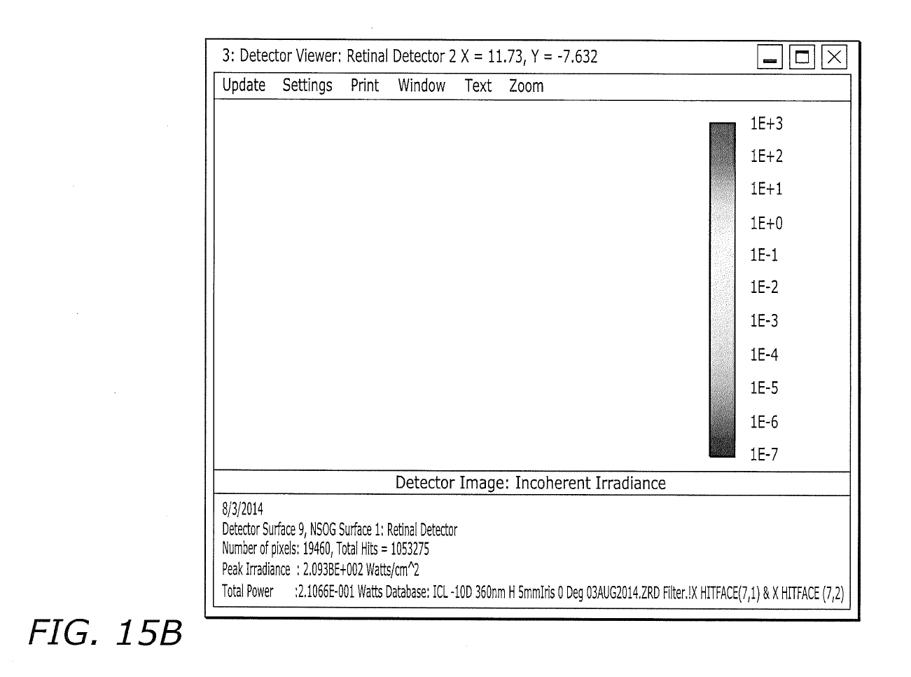

FIGS. 15A-B show the rays that hit the retina, but do not hit the walls of the hole. Referring to FIG. 15A, a ray trace analysis showing a side view of the rays hitting the retina is shown. Referring to FIG. 15B, a ray trace analysis illustrating the rays of FIG. 15A hitting the retina from a front view is shown. These are the rays that either hit the optical zone or pass through the central hole without hitting the walls of the hole or any other structure. It can be seen from these figures that the rays produce a well formed image, represented by the small spot on the retina, most easily seen in FIG. 15B.

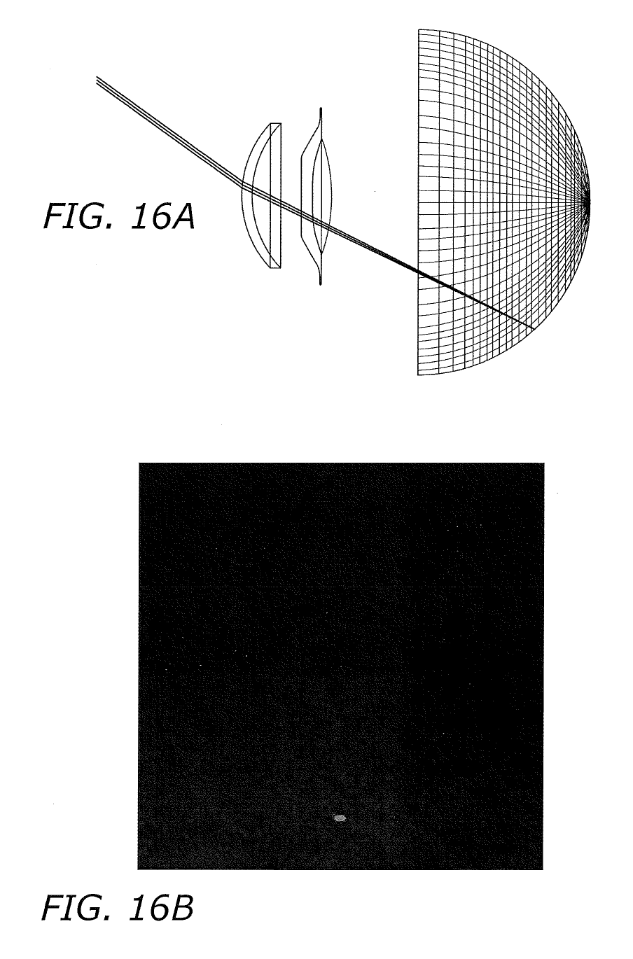

FIGS. 16A-B show the rays that hit the retina, but did not hit any part of the ICL surface. Referring to FIG. 16A, a ray trace analysis showing a side view of the rays hitting the retina is shown. Referring to FIG. 16B, a ray trace analysis illustrating the rays of FIG. 16A hitting the retina from a front view is shown. These are the rays that passed straight through the central hole of the ICL.

It is therefore possible to identify clearly where each ray that hit the retina comes from. First we study the effect of the hole diameter on the amount of scattered light. From a purely fluid-flow point of view, the hole could be as small as 50 um (0.05 millimeters), as discussed by B. W. Fleck in “How large must an iridotomy be?”, British Journal of Ophthalmology, 74, 583-588, 1990, which is hereby incorporated in its entirety herein, although it would be advantageous to make the hole much larger, to give enough margin for fluid flow and avoid potential problems of occlusion. For example, eye inflammations or other physiological processes might produce particles that could clog a small hole. Therefore, from the fluid-flow point of view, it would be advantageous to make the hole as large as possible.

From a fluid flow point of view, the best location for the hole is at the lens center. Other embodiments, however, are possible that may place smaller holes at different points of the optic or even outside the optical region, as discussed above.

From an optical point of view on the other hand, it is better to make the hole as small as possible or better yet, not to have a hole at the lens center. The reason for this is that the internal walls of the hole can scatter light, as discussed above, that may result in perceived halos by the lens wearer.

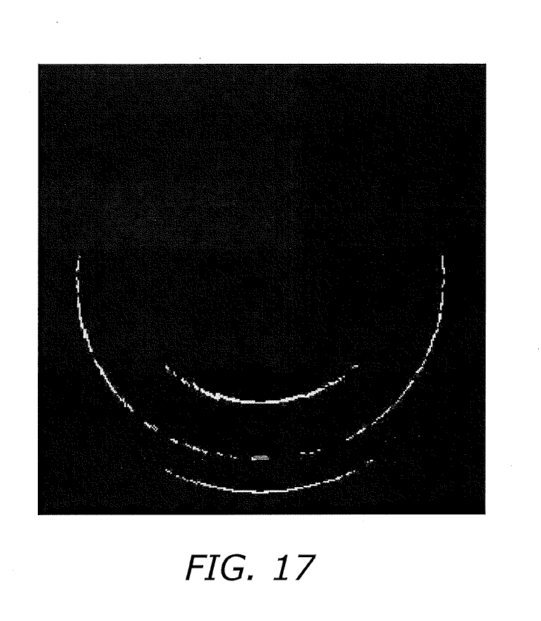

Full non-sequential ray trace analysis of the light scattering by a center hole varying in diameter from 0.10 millimeters to 0.360 millimeters was performed. FIG. 17 presents the ray tracing analysis, from a front view, for the case having a 0.30 millimeter diameter central hole, considering a pupil diameter of 4.2 millimeters and with a light incidence of 35 degrees. FIG. 17 shows all the rays that hit the retina, which in this case was 821,635 rays, giving 164.33 mW and 123.65 W/cm² of peak irradiance. The rays that go through the ICL lens form a small spot at the bottom center of the image, while the rays that hit the internal walls of the hole form the arcs that appear in the image.

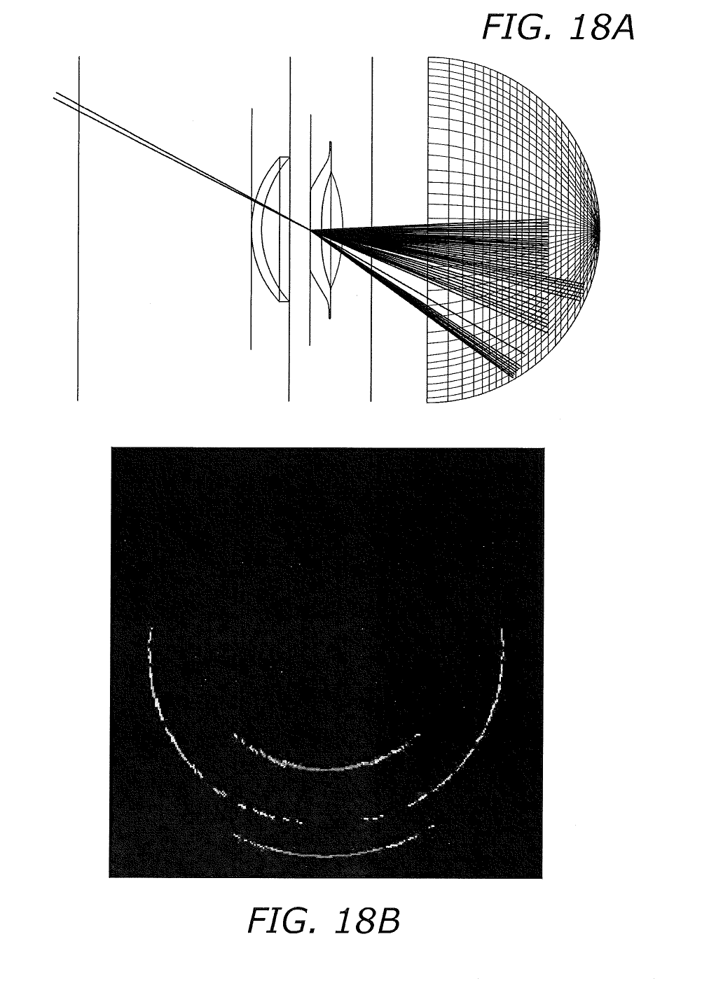

Since the lens was divided into separate faces, as discussed with reference to FIGS. 10A-C, it is possible to filter the rays according to which face of the rays that the rays encounter. FIGS. 18A-B show all the rays that hit the walls of the hole, forming arcs. Referring to FIG. 18A, a ray trace analysis showing a side view of the rays hitting the retina is shown. Referring to FIG. 18B, a ray trace analysis illustrating the rays of FIG. 18A hitting the retina from a front view is shown. A total of 313 rays do not hit any lens surface, that is, the rays go through the hole and form a small spot on the same position as the rays that go through the optical zone. The table below summarizes these results:

TABLE I
Light scattering by a 300 um hole. The hole walls
satter only 0.030% of the peak irradiance.
Peak Irradiance (W/cm2): 123.570
Total Power (mW): 164.120
Hits: 820590

| LENS SURFACE | | HIT ICL SURFACE | | | | |
|---|---|---|---|---|---|---|
| — | | Peak Irr. | Power | Hits | % Power | % Irr. |
| — | FACE | (W/cm2) | (mW) | — | (%) | (%) |
| PLATE HAPTIC | 0 | 0.000 | 0.000 | 0 | 0.000 | 0.000 |
| HOLE WALLS | 1 | 0.037 | 0.645 | 3225 | 0.393 | 0.030 |
| OPTIC | 2 | 123.560 | 162.350 | 811763 | 98.922 | 98.690 |
| THROUGH THE HOLE | — | 1.603 | 1.120 | 5602 | 0.682 | 1.280 |
| | | | | TOTAL | 100.0 | 100.0 |

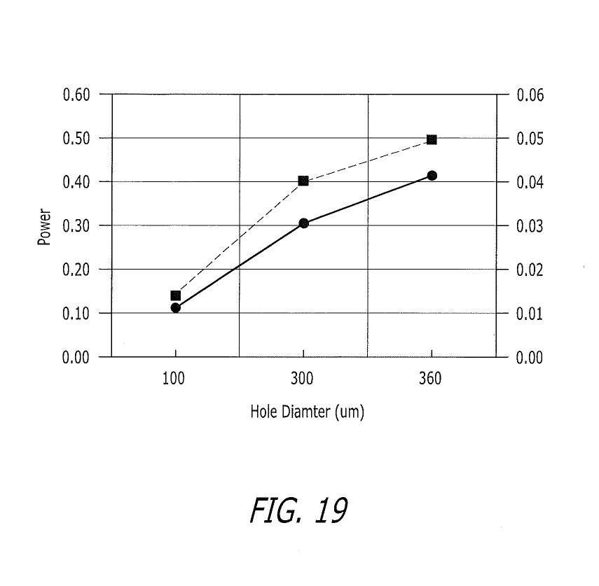

Tables II and III show similar results for a 360 um hole and a 100 um hole and FIG. 19 plots the resulting percentage power and peak irradiance scattered by the hole walls versus hole diameter. This plot clearly shows that, from an optical point of view, one preferred embodiment is to have the hole diameter as small as possible, to minimize light scattering.

TABLE II
Light scattering by a 360 um hole.
TOTAL LIGHT THAT ENTERED THE 4.2 mm PUPIL AT 15 DEGREES:
Peak Irradiance (W/cm2): 123.930
Total Power (mW): 164.650
Hits: 823272

| LENS SURFACE | | HIT ICL SURFACE | | | | |
|---|---|---|---|---|---|---|
| — | | Peak Irr. | Power | Hits | % Power | % Irr. |
| — | FACE | (W/cm2) | (mW) | — | (%) | (%) |
| PLATE HAPTIC | 0 | 0.000 | 0.000 | 0 | 0.000 | 0.000 |
| HOLE WALLS | 1 | 0.050 | 8.060E−01 | 4032 | 0.490 | 0.040 |
| OPTIC | 2 | 123.920 | 162.060 | 810308 | 98.427 | 97.946 |
| THROUGH THE HOLE | — | 2.548 | 1.786 | 8932 | 1.085 | 2.014 |
| | | | | TOTAL | 100.0 | 100.0 |

TABLE III
Light scattering by a 100 um hole.
Peak Irradiance (W/cm2): 123.650
Total Power (mW): 164.330
Hits: 821635

| LENS SURFACE | | HIT ICL SURFACE | | | | |
|---|---|---|---|---|---|---|
| — | | Peak Irr. | Power | Hits | % Power | % Irr. |
| — | FACE | (W/cm2) | (mW) | — | (%) | (%) |
| PLATE HAPTIC | 0 | 0.000 | 0.000 | 0 | 0.000 | 0.000 |
| HOLE WALLS | 1 | 0.014 | 0.226 | 1129 | 0.137 | 0.011 |
| OPTIC | 2 | 123.650 | 164.040 | 820.193 | 99.824 | 99.916 |
| THROUGH THE HOLE | — | 0.090 | 0.063 | 313 | 0.038 | 0.073 |
| | | | | TOTAL | 100.0 | 100.0 |

In the next study, the shape of the hole was modified and the effect of this change on the resulting halos and arcs reaching the retina was evaluated. The central hole diameter was fixed at 360 um (0.360 millimeters) and the optical zone diameter was set to 5 mm. The hole shape was modified by tilting the walls by varying amounts. Thus, instead of having a cylindrical shape, the hole becomes a truncated cone. FIG. 6 illustrates the basic design studied. In this example, the hole walls were tilted by 45 degrees. In the light scattering simulations that follow, only the rays forming arcs and halos and those forming the central spot at the lower center of each figure are displayed.

Staring with the simplest possible case, where there is no hole, provides a baseline to compare the results across the simulations. In the figures that follow light scattering results for the “no hole” case, as well as the “zero angle” or straight walls, 5 degrees tilted walls, 10 degrees, 15, 35, 45, 55, 65 and 75 degrees cases are presented. In all cases the lens is a −10D ICL, with pupil diameter of 5 mm and the light angle of incidence is 35 degrees. Except for the “no hole” case, the hole diameter is 360 um (0.360 millimeters) at the anterior lens surface.

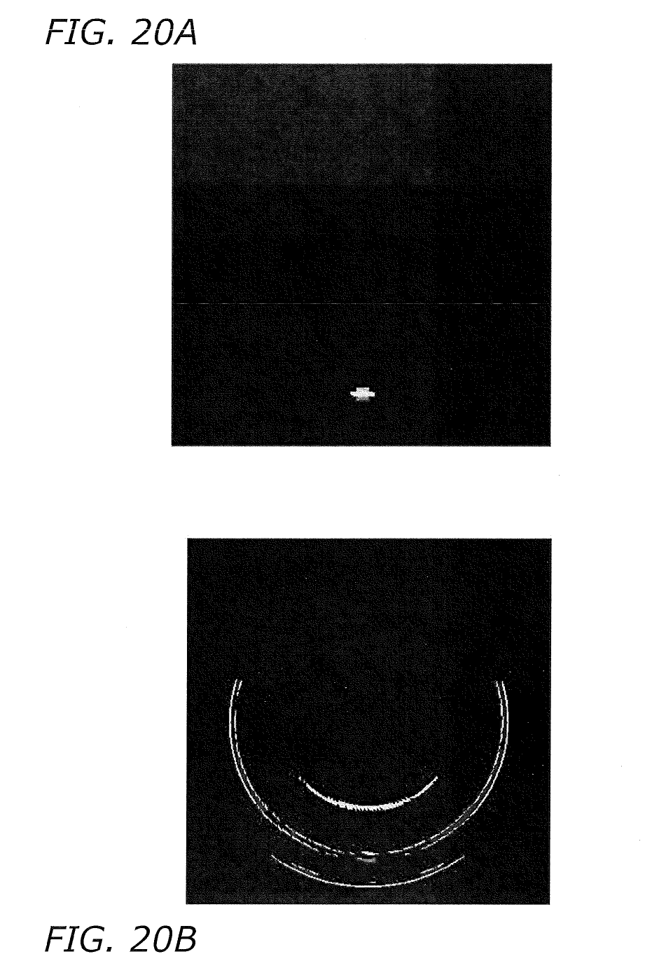

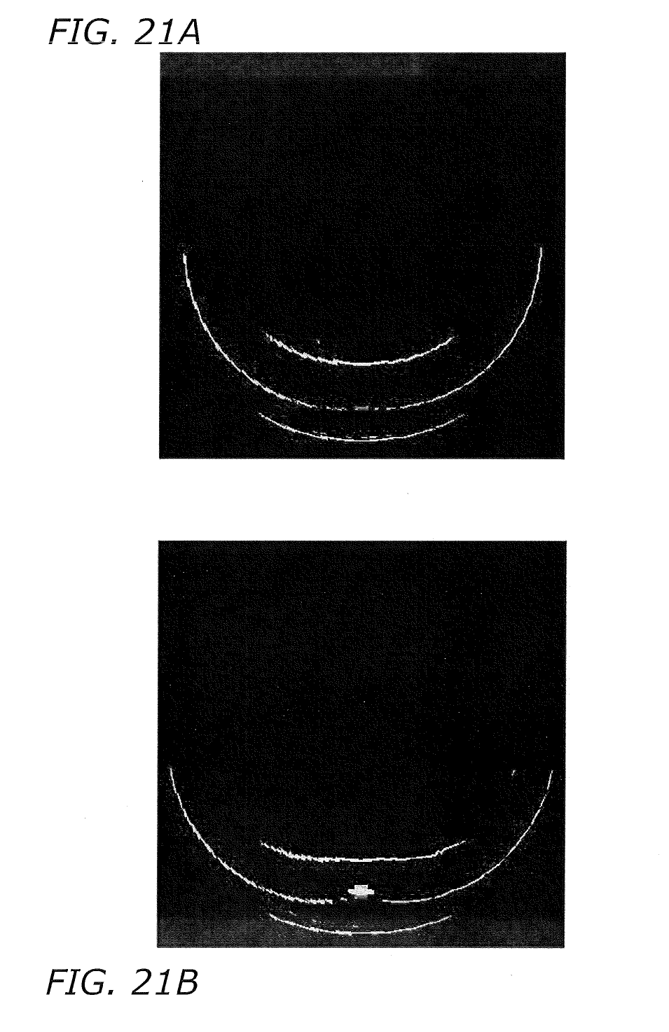

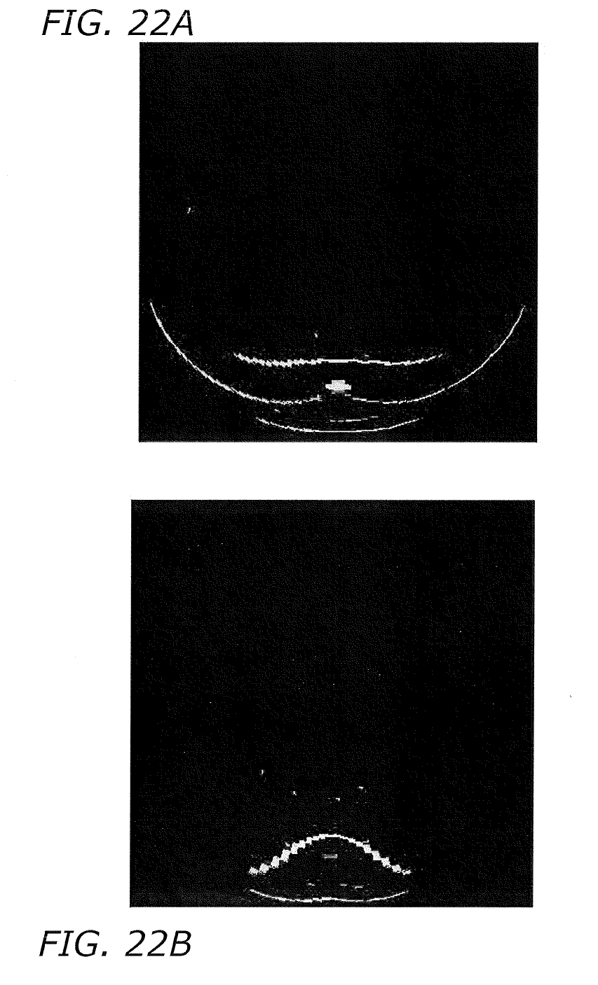

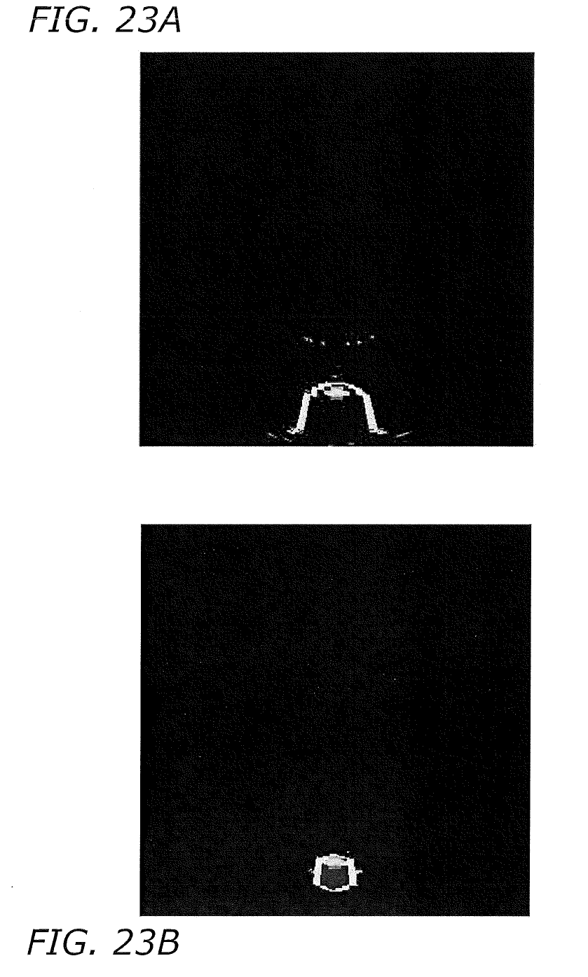

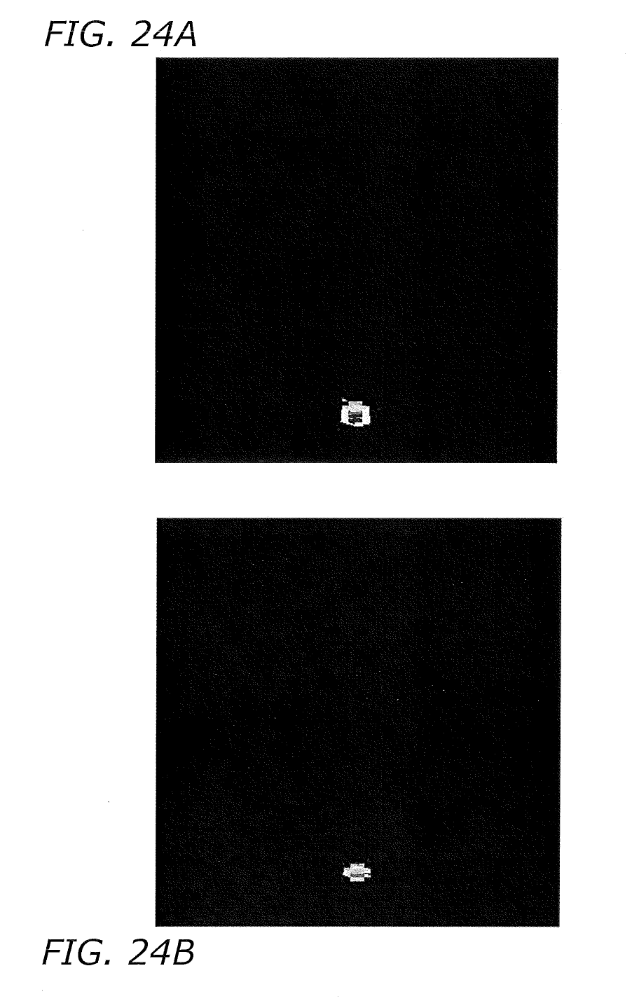

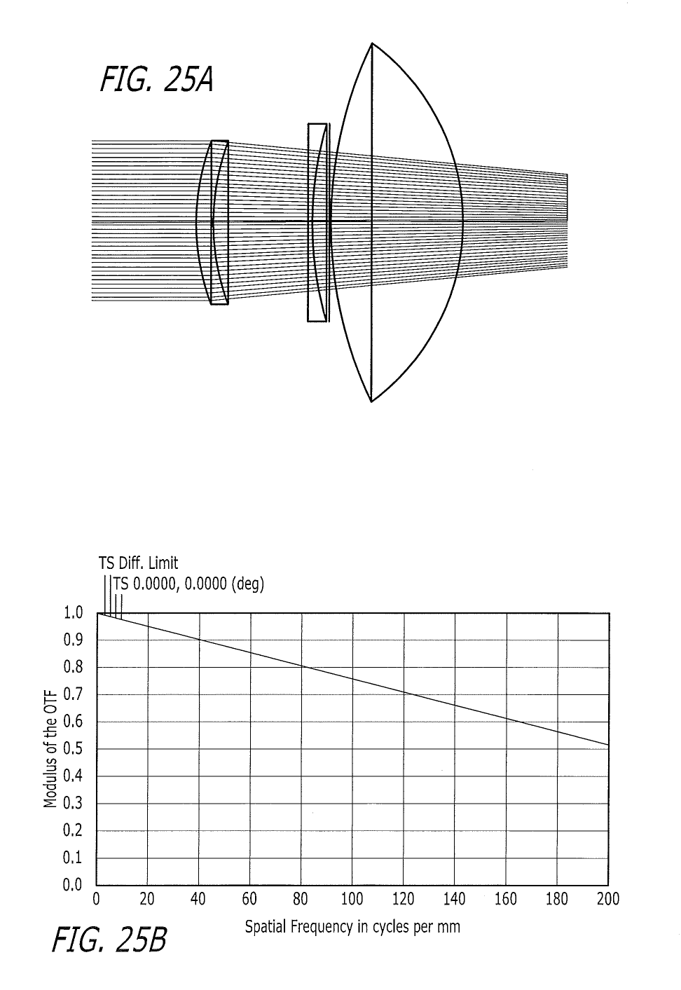

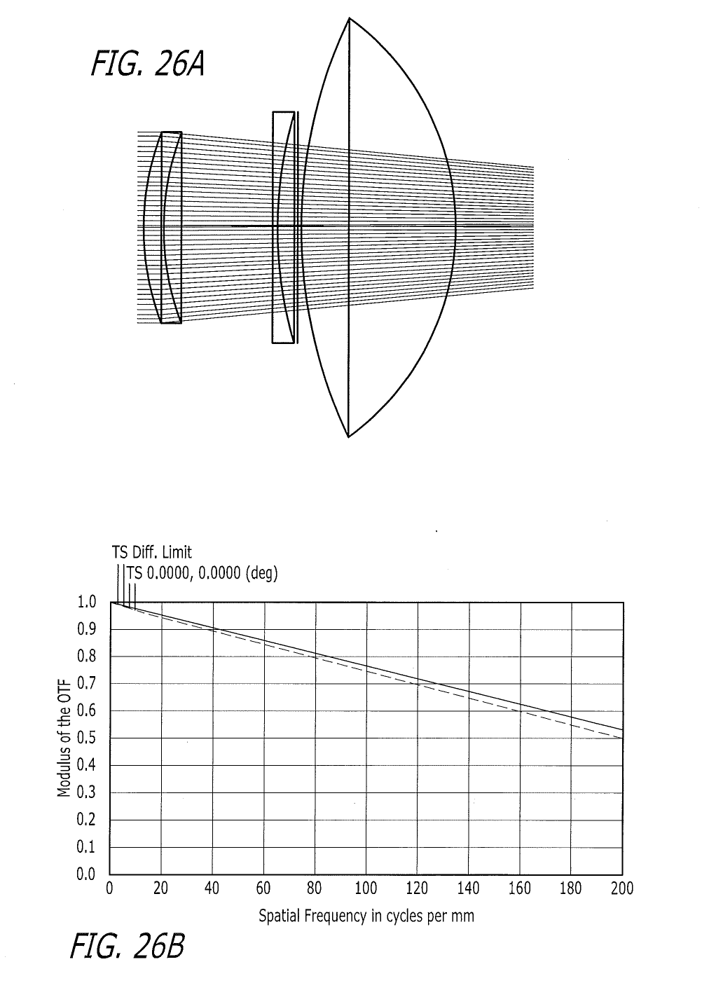

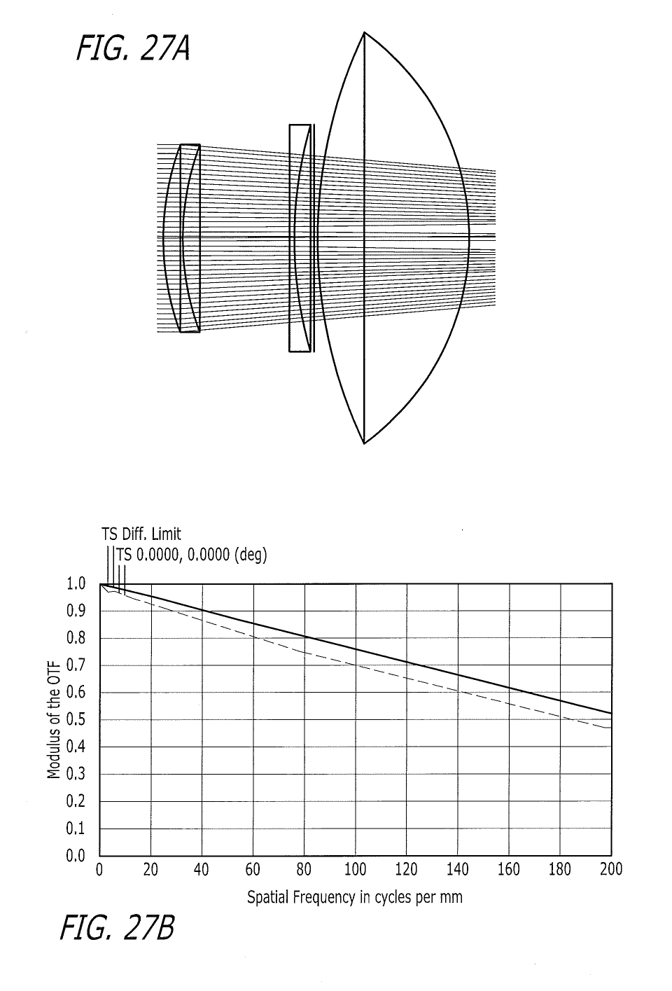

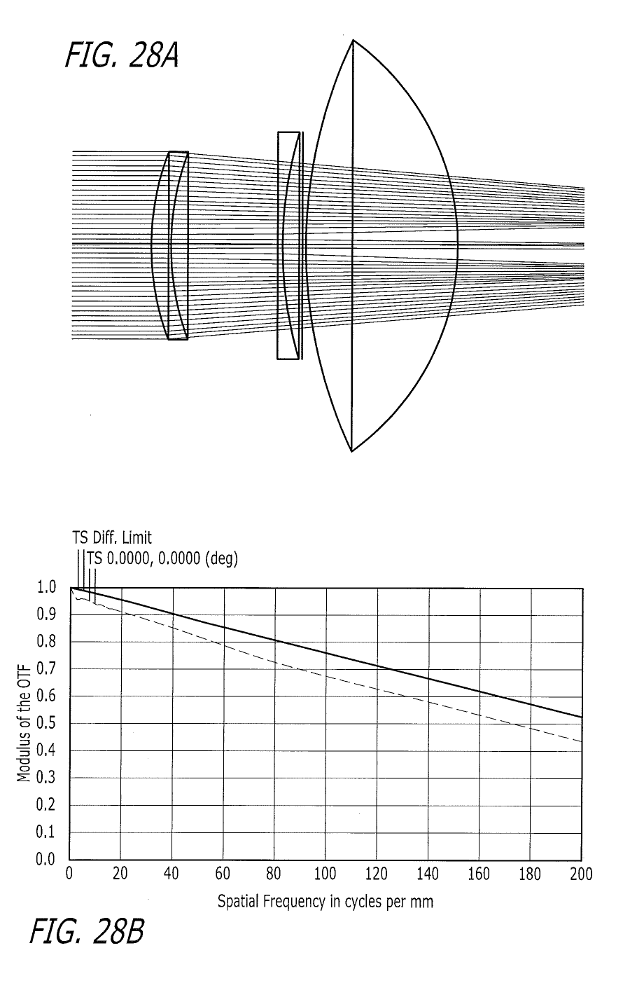

FIGS. 20A-B to FIGS. 24A-B present the results of the study showing how the light scattering varies as a function of the angle of the walls of the hole. Referring to FIGS. 25A, 26A, 27A, 28A and 29A, ray trace analyses showing a side view of the rays hitting the retina is shown. In particular, the images of FIG. 20A (the “no hole” case) and FIG. 24B (the “75 degree tilt” case) are remarkably similar. This means that by tilting the hole walls, it is possible to make the arcs and halos collapse back on top of the original image formed by the light passing through the optical zone of the lens. The lens with the 360 um (0.36 millimeters) central hole, having its walls tilted 75 degrees, does not produce arcs and halos and is virtually identical to the “no hole” case.

The explanation for this is that as the angle of the hole walls is increased, the light rays going through the optic and hitting the hole wall encounter the hole wall at a steeper angle and no longer undergo total internal reflection. These rays simply go through the wall and fall upon the retina with a very small deviation, at approximately the same location as the light passing through the rest of the optical zone. For those rays that hit the hole wall from the interior of the hole (these rays don't hit the optical zone) a similar situation occurs. When the wall is tilted sufficiently, the wall gets “out of the way” and the rays no longer encounter the wall, passing straight through the hole and falling upon the retina. Therefore, by tilting the hole walls arcs and halos may be effectively eliminated.

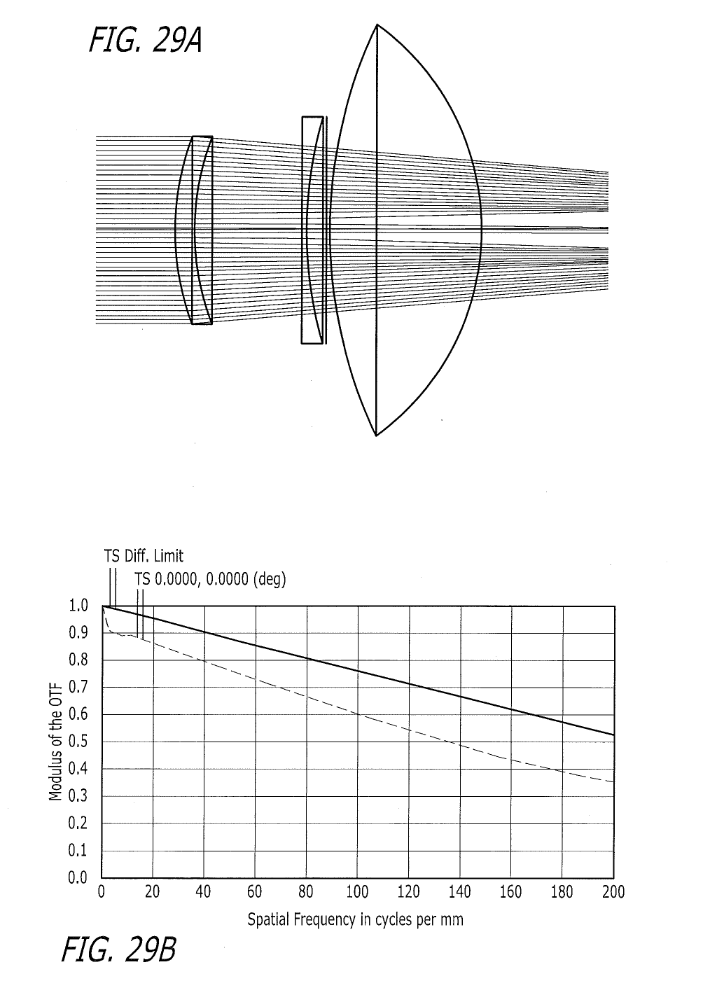

Having demonstrated that the tilted hole walls can solve the light scattering problems identified above, it is also possible to investigate whether this has any adverse effects on the lens optical properties, as measured by the Modulation Transfer Function (MTF). FIGS. 25A-B to FIGS. 29A-B show the MTF for the “no hole” case, as well as the zero degrees, 55, 65 and 75 degrees cases. For each of the Figures, FIG. nA illustrates the layout and ray trace for that layout, while FIG. nB illustrates the MTF graph; in each case, “n” is the Figure number.

FIG. 17 show that for the “no hole” case, MTF is diffraction limited, as would be expected. The MTF degradation of each successive case is small, and even where in the “75 degree” case, the MTF degradation, while seemingly large, is still acceptable and within the limit set by the ISO 11979-2 standard.

Although the results above are promising, they don't provide for the case where the angle of incidence of the light is restricted to the 35 degrees angle of incidence. Therefore, in to provide for the worst case, further testing was done using a hole with 75 degrees tilted walls and varying the light angle of incidence from 5 degrees to 45 degrees.

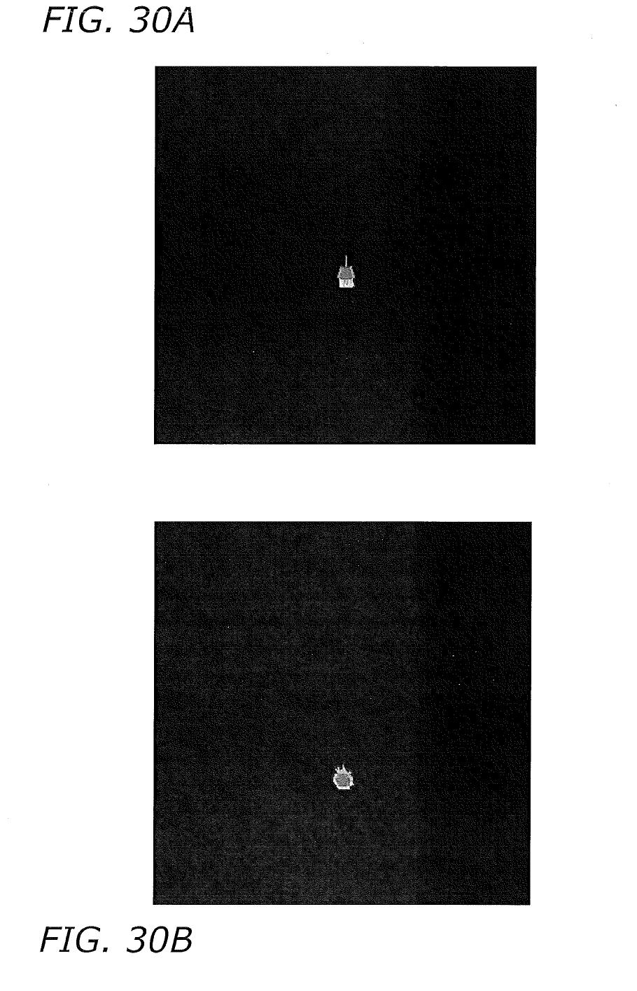

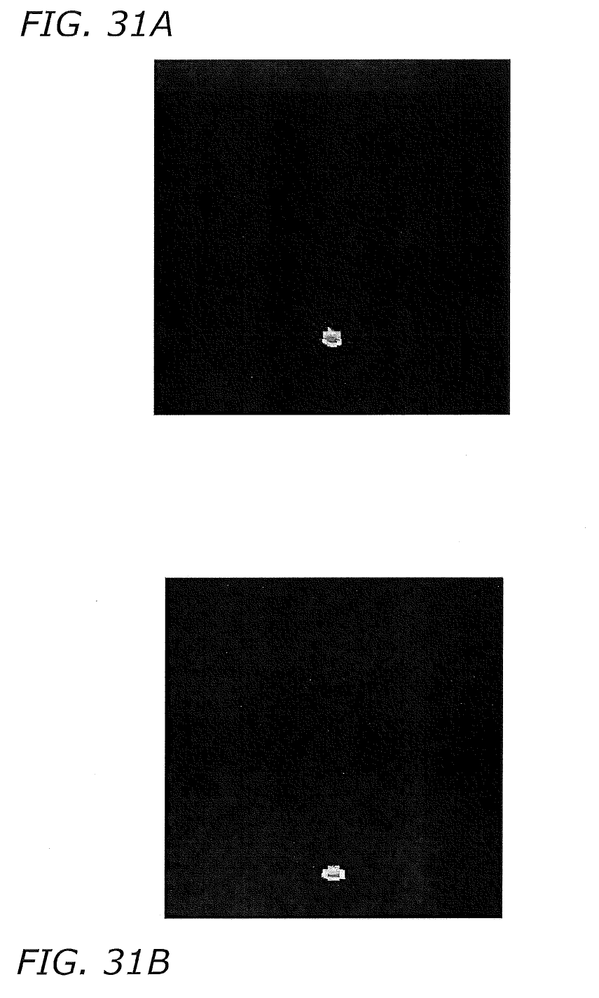

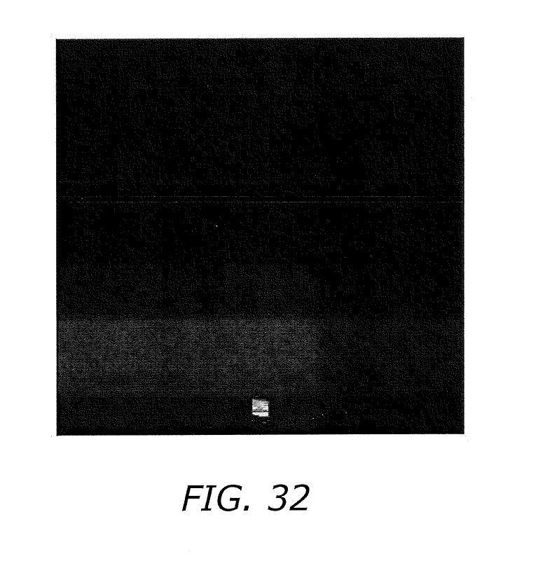

FIGS. 30A-B to FIG. 32 illustrates the ray trace analysis using a −10D ICL lens, with a central hole of 360 um (0.360 millimeters) diameter, with walls tilted 75 degrees from the anterior surface to the posterior surface of the optical zone of the ICL. The pupil diameter was set at 5 mm. The angle of incidence of the light was changed as indicated in each Figure.

Notice that all figures show the rays converging to the same area of the retina and no halos or arcs are present. This shows that the angle of incidence of the light can be varied from 5 degrees to 45 degrees without producing halos or arcs. Similar results have been achieved for holes with walls tilted by 55 and 65 degrees.

While several forms of the invention have been illustrated and described, it will be apparent that various modifications can be made without departing from the spirit and scope of the invention.

We claim:

1. An intraocular contact lens for implantation into an eye comprising:
a body portion surrounding an optical zone having a thickness and an optical axis transverse to a longitudinal axis of the body portion; and
a hole disposed in the optical portion extending through the thickness of the optical zone from an anterior surface of the optical zone to a posterior surface of the optical zone, the hole having a wall formed by the thickness of the optical zone, the hole wall being angled relative to the optical axis such that a diameter of the anterior surface of the hole is different than a diameter of the posterior surface.

2. The lens of claim 1, wherein the diameter of the anterior surface is smaller than the diameter of the posterior surface,

3. The lens of claim 1, wherein the diameter of the anterior surface is larger than the diameter of the posterior surface.

4. The lens of claim 1, wherein the hole wall is angled relative to the optical axis in a range of 5 degrees to 75 degrees.

5. The lens of claim 1, wherein the hole wall is angled 65 degrees relative to the optical axis.

6. The lens of claim 1, wherein the hole wall has a curvature extending between the anterior surface and the posterior surface.

7. The lens of claim 6, wherein the curvature has a 2.0 millimeter radius.

8. The lens of claim 1, wherein the hole wall further comprises (i) an annular portion extending from the anterior surface for a selected distance to an endpoint and (ii) a tapered portion extending from the endpoint to the posterior surface,

9. The lens of claim 8, wherein a diameter of the hole within the annular portion is smaller than the diameter of the hole at the posterior surface.

10. The lens of claim 1, wherein the hole wall has a step-like profile with each step having a larger diameter than a next adjacent step moving in a direction from a step having a smallest diameter to a step having a largest diameter.

11. The lens of claim 1, wherein the hole is disposed in a center of the optical zone.

12. The lens of claim 1, wherein the hole is disposed in the body portion near a transition between the body portion and the optical zone,

13. The lens of claim 12, wherein a plurality of holes are disposed in the body portion near a transition between the body portion and the optical zone.

14. The lens of claim 1, wherein a plurality of holes are disposed in the optical zone.

15. The lens of claim 1, wherein the diameter of the anterior surface is 300 microns and a wall tilt from the anterior surface to the posterior surface is 65 degrees.

16. The lens of claim 1, wherein the diameter of the anterior surface is within 50 microns to 400 microns and a wall tilt from the anterior surface to the posterior surface is within 50 degrees to 75 degrees.

17. A method of forming a tapered hole configured to reduce an amount of light scattered by a wall of the tapered hole in an optical zone of an intraocular contact lens, the method comprising:
drilling a hole through the optical zone of the intraocular contact lens, the optical zone having an anterior surface and a posterior surface; and
using a conical shaped drill shape walls of the hole such that the tapered hole is formed, wherein the tapered hole has a diameter at the anterior surface of the optical zone and a diameter at the posterior surface of the optical zone.

18. The method of claim 17, wherein the diameter of the anterior surface is 300 microns and a wall tilt from the anterior surface to the posterior surface is 65 degrees.

19. The method of claim 17, wherein the diameter of the anterior surface is smaller than the diameter of the posterior surface.

20. The method of claim 17, wherein the diameter of the anterior surface is larger than the diameter of the posterior surface.
